(19) United States
(12) Patent Application Publication
(10) Pub. No.: US 2019/0184209 A1
(43) Pub. Date: Jun. 20, 2019

(54) HAIR TREATMENT AGENT WITH ANTI-DANDRUFF ACTION

(71) Applicant: Henkel AG & Co. KGaA, Duesseldorf (DE)

(72) Inventors: Thomas Schroeder, Hamburg (DE); Rene Krohn, Norderstedt (DE)

(73) Assignee: Henkel AG & Co. KGaA, Duesseldorf (DE)

(21) Appl. No.: 16/219,046

(22) Filed: Dec. 13, 2018

(30) Foreign Application Priority Data
Dec. 18, 2017 (DE) 10 2017 223 063.0

(51) Int. Cl. A61Q 5/00; A61Q 5/02; A61K 8/36; A61K 8/44

(52) U.S. Cl. CPC A61Q 5/006; A61K 8/44; A61K 8/361; A61Q 5/02

(57) ABSTRACT

The present disclosure concerns a hair treatment agent that contains, in a cosmetic support—with respect to its weight—
a) from about 0.05 to about 5.0% by weight of zinc oxide,
b) from about 0.05 to about 3.00% by weight of at least one anti-dandruff substance that is different from a),
c) from about 0.10 to about 10.00% by weight of at least one saturated or unsaturated, straight-chained or branched C₁₀-C₂₄ carboxylic acid, and
d) from about 0.01 to about 5.00% by weight of at least one amino acid.

### CROSS-REFERENCE TO RELATED APPLICATION

This application claims priority to German Patent Application No. 10 2017 223 063.0, filed Dec. 18, 2017, which is incorporated herein by reference in its entirety.

### TECHNICAL FIELD

The present disclosure concerns the field of cosmetic and relates to hair treatment agents that contain zinc oxide, a further anti-dandruff substance, a fatty acid as well as an amino acid in a cosmetic support as a combination of substances.

The present disclosure furthermore relates to the cosmetic use of the combination of substances to increase the anti-dandruff effectiveness of hair treatment agents, as well as to a cosmetic method for removing, reducing, alleviating and/or preventing dandruff.

### BACKGROUND

Combatting dandruff constitutes a vital aspect in cosmetic hair treatment, because even mild flaking of the scalp or other hirsute regions of the body is taken as an indication of inadequate care and hygiene. Furthermore, dandruff is associated with distressing itching, which induces a scratch reaction and can lead to injuries to the affected parts of the skin. In turn, skin injuries can frequently form the basis of infections and diseases.

The formation of dandruff can be promoted by a variety of factors such as genetic predisposition, a tendency to over-produce oil (seborrhea), hormonal fluctuations, stress, climatic conditions or incorrect hair care, for example. However, the primary contribution to the formation of dandruff is the colonization of the scalp with yeast fungi of the genus Malassezia.

Thus, combatting dandruff focuses on reducing and/or removing the colonization of the scalp by Malassezia.

The prior art contains a multitude of cosmetically acceptable and effective anti-dandruff substances which are usually incorporated into shampoos or tonics for treating the scalp. Examples of frequently used anti-dandruff substances are piroctone olamine, climbazole, zinc pyrithione and other zinc salts, special plant extracts, sulfur and/or selenium sulfide.

DE 102011079539 and DE 102014225083 disclose cosmetic hair treatment agents with good anti-dandruff effectiveness, which contain organic or inorganic zinc salts as well as optional other anti-dandruff substances.

Nevertheless, on the one hand there is still a need for hair treatment agents with even more improved anti-dandruff effectiveness, which is evident from the first application, is long-lasting and sustainable; and on the other hand, stabilization of the anti-dandruff substances, which are frequently insoluble in water and/or surfactant solutions, is undesirably expensive.

### BRIEF SUMMARY

The present disclosure therefore provides a hair treatment agent which contains, in a cosmetic support—with respect to its weight—

a) from about 0.05 to about 5.0% by weight of zinc oxide,
b) from about 0.05 to about 3.00% by weight of at least one anti-dandruff substance that is different from a),
c) from about 0.10 to about 10.00% by weight of at least one saturated or unsaturated, straight-chained or branched C₁₀-C₂₄ carboxylic acid, and from about 0.01 to about 5.00% by weight of at least one amino acid.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The objective of the present disclosure is to provide hair treatment agents that are stable upon storage, with which dandruff can be quickly, effectively and sustainably removed, reduced, alleviated and/or prevented.

In addition, the agents should be capable of being produced in as energy and cost-effective manner as possible and should exhibit outstanding care properties.

It has now surprisingly been discovered that the objectives specified above can be achieved in an outstanding manner by employing hair treatment agents that contain zinc oxide, a further anti-dandruff substance, a fatty acid as well as an amino acid as a combination of substances.

Compared with conventional anti-dandruff agents—usually based on zinc pyrithione—the hair treatment agents exhibit an improved anti-dandruff effectiveness. At the same time, they are extremely compatible with the skin and endow the hair with skincare benefits following treatment.

In a first aspect, the present disclosure therefore provides a hair treatment agent which contains, in a cosmetic support—with respect to its weight—

a) from about 0.05 to about 5.0% by weight of zinc oxide,

b) from about 0.05 to about 3.00% by weight of at least one anti-dandruff substance that is different from a),
c) from about 0.10 to about 10.00% by weight of at least one saturated or unsaturated, straight-chained or branched C₁₀-C₂₄ carboxylic acid, and
d) from about 0.01 to about 5.00% by weight of at least one amino acid.

The hair treatment agents as contemplated herein contain the substances a) to d) in a cosmetic support. In the context of the present disclosure, this should be understood to mean an aqueous or aqueous alcoholic support.

The cosmetic support contains at least about 60% by weight, for example at least about 65% by weight, for example at least about 70% by weight and for example at least about 75% by weight of water (with respect to the total weight of the agent).

Furthermore, the cosmetic support may contain from about 0.01 to about 20% by weight, preferably from about 0.05 to about 15% by weight and in particular from about 0.1 to about 10% by weight of at least one alcohol.

Examples of suitable alcohols are ethanol, 1-propanol, 2-propanol, isopropanol, glycerine, diglycerine, triglycerine, 1-butanol, 2-butanol, 1-pentanol, 2-pentanol, 1,2-propanediol, 1,2-butanediol, 1,2-pentanediol, 1,5-pentanediol, 1-hexanol, 2-hexanol, 1,2-hexanediol, 1,6-hexanediol, polyethyleneglycols, sorbitol, sorbitan, benzyl alcohol, phenoxyethanol or mixtures of these alcohols.

Water-soluble alcohols are suitable.

Ethanol, 1,2-propanediol, glycerine, benzyl alcohol and/or phenoxyethanol as well as mixtures of these alcohols are suitable.

The hair treatment agents as contemplated herein contain from about 0.05 to about 5.0% by weight of zinc oxide (with respect to the total weight of the agent) as the first included component.

Zinc oxide is used in the hair treatment agents as contemplated herein (with respect to the total weight of the agent) in a quantity of from about 0.10 to about 4.00% by weight, for example from about 0.25 to about 3.00% by weight and in particular of from about 0.50 to about 2.00% by weight.

In the compositions as contemplated herein, zinc oxide acts as a booster for the anti-dandruff substance b). Zinc oxide is essentially insoluble in water and has to be stabilized in a water and/or surfactant base so that the stability and/or homogeneity of the composition as a whole is maintained. This problem principally arises in a pH range of <9 and in the context of the present disclosure, is solved by adding the acids c) and d). As a result, the addition of—in particular polymeric—thickening agents is not necessary.

The term “insoluble in water” as used in the context of the present application should be understood to mean that at about 20° C., less than about 1 g of the relevant compound (ZnO, anti-dandruff substance b)) is soluble in about 1 L of water. For example, less than about 100 mg, for example less than about 50 mg and in particular less than about 25 mg of the relevant compound (ZnO, anti-dandruff substance b)) is soluble in about 1 L of water at about 20° C.

The hair treatment agents as contemplated herein contain, as the second included component, from about 0.05 to about 3.0% by weight (with respect to the total weight of the agent) of an anti-dandruff substance b) that is different from ZnO.

Anti-dandruff substances b) that are soluble in a surfactant solution and/or soluble in water are used as the anti-dandruff substances b) in the hair treatment agents as contemplated herein. These may be selected from piroctone olamine, climbazole, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts and/or arnica extracts.

In the context of the present disclosure, piroctone olamine and/or climbazole is for example used as the anti-dandruff substance. Piroctone olamine is suitable.

It has been discovered that ZnO reacted with the acids c) and d) in combination with piroctone olamine and/or climbazole can be used to formulate stable, highly effective anti-dandruff shampoos.

The anti-dandruff substance(s) b) is (are) used in the hair treatment agents as contemplated herein in a quantity of from about 0.10 to about 2.50% by weight, for example from about 0.15 to about 2.00% by weight and in particular from about 0.20 to about 1.50% by weight (with respect to the total weight of the hair treatment agent). An anti-dandruff substance b) that is soluble in a surfactant solution or soluble in water, for example piroctone olamine and/or climbazole and in particular piroctone olamine, is used in the quantities specified above.

As the third included component, the hair treatment agents as contemplated herein contain from about 0.10 to about 10.00% by weight of at least one saturated or unsaturated, straight-chained or branched C₁₀-C₂₄ carboxylic acid (with respect to the total weight of the agent).

Saturated or unsaturated, straight-chain or branched fatty acids containing from about 12 to about 22, for example from about 14 to about 20 and in particular from about 16 to about 18 carbon atoms in the carbon chain are preferred, such as, for example, isostearic acid, isopalmitic acid, 2-ethylhexanoic acid, capric acid, lauric acid, isotridecanoic acid, myristic acid, palmitic acid, palmitoleic acid, stearic acid, oleic acid, elaidic acid, petroselinic acid, linoleic acid, linolenic acid, eleostearic acid, arachidic acid, gadoleic acid, behenic acid and erucic acid as well as technical mixtures thereof.

Fatty acid cuts which are obtained from coconut oil or palm oil are suitable; palmitic acid, stearic acid or mixtures of these acids are suitable.

In one embodiment, the hair treatment agents as contemplated herein contain (with respect to their total weight) from about 0.50 to about 8.00% by weight, preferably from about 1.00 to about 6.00% by weight and in particular from about 1.50 to about 5.00% by weight of at least one saturated or unsaturated, straight-chained or branched C₁₀-C₂₄ carboxylic acid, in particular palmitic acid and/or stearic acid.

Finally, the hair treatment agents as contemplated herein contain from about 0.01 to about 5.00% by weight of at least one amino acid as the fourth essential component.

In principle, the term “suitable amino acids” as used in the context of the present disclosure should be understood to mean all physiologically acceptable amino acids as well as their salts.

However, for technical formulation reasons, it is suitable for the amino acid(s) d) used to be at least one non-essential amino acid.

In the context of the present disclosure, alanine, aspartic acid, cysteine, cystine, glutamic acid, glycine, proline, serine and/or tyrosine are suitable; glycine, proline and/or serine are given note, and glycine is exemplary.

The amino acid(s) d) are preferably used in the hair treatment agents as contemplated herein in a quantity of from about 0.02 to about 4.00% by weight, for example from about 0.05 to about 3.00% by weight and in particular from about 0.10 to about 2.00% by weight (with respect to the total weight of the hair treatment agent). Alanine, aspartic acid, cysteine, cystine, glutamic acid, glycine, proline, serine and/or tyrosine and in an embodiment glycine are used in the quantities specified above.

The hair treatment agents as contemplated herein may be present as a hair cleaning agent, hair conditioner, hair mask, hair styling agent, hair tonic, etc.

Hair treatment agents as contemplated herein that are formulated as a hair shampoo and/or as a hair tonic are preferred.

Hair treatment agents as contemplated herein that are in the form of a shampoo preferably comprise at least one foaming surfactant, in addition to the substances a) to d). When selecting the surfactants and when producing the shampoo, care must be taken that cleaning the hair with the shampoo does not irritate or stress the scalp.

Thus, hair shampoos that have been shown to be suitable are those that have a pH which is slightly acidic, and/or which contain a limited quantity of surfactant and/or which comprise a mixture of particularly mild (to the skin) types of surfactant.

Hair shampoos with a pH in the range from about 4.0 to about 6.0, for example from about 4.2 to about 5.8, for example from about 4.3 to about 5.6 and in an embodiment from about 4.5 to about 5.5 are suitable.

A maximum total surfactant content of about 20% by weight—with respect to the weight of the hair treatment agent as contemplated herein (shampoo)—is suitable. A maximum total surfactant content of about 18.00% by weight, for example a maximum of about 16.00% by weight and for example a maximum of about 15.00% by weight is used in an embodiment.

A mixture of at least one anionic surfactant e) and at least one amphoteric/zwitterionic surfactant f) has been shown to be a particularly mild surfactant mixture for the hair treatment agents (shampoos) as contemplated herein.

In an embodiment, the hair treatment agents as contemplated herein are shampoos and additionally contain at least one anionic surfactant e) and at least one amphoteric and/or zwitterionic surfactant f). Optionally, the hair shampoos may furthermore contain a non-ionic surfactant g).

Examples of suitable anionic surfactant types e) that may be used in the hair treatment agents (shampoos) as contemplated herein are:

ethercarboxylic acids with formula R—O—(CH₂—CH₂O)ₓ—CH₂—COOH, in which R is a linear or branched, saturated or unsaturated alkyl group containing from about 8 to about 30 C atoms and x=0 or from about 1 to about 16,

acyl sarcosides containing from about 8 to about 24 C atoms in the acyl group,

acyl taurides containing from about 8 to about 24 C atoms in the acyl group,

acyl isethionates containing from about 8 to about 24 C atoms in the acyl group,

sulfosuccinic acid mono- and/or -dialkylesters containing from about 8 to about 24 C atoms in the alkyl group and sulfosuccinic acid monoalkylpolyoxyethylesters containing from about 8 to about 24 C atoms in the alkyl group and from about 1 to about 6 oxyethyl groups,

alpha-olefin sulfonates containing from about 8 to 2 about 4 C atoms,

alkylsulfates and/or alkylethersulfate salts with formula R—(OCH₂—CH₂)ₙ—O—SO₃X, in which R preferably represents a straight-chain or branched, saturated or unsaturated alkyl group containing from about 8 to about 30 C atoms, x represents the number 0 or from about 1 to about 12 and X represents an alkali, alkaline earth, ammonium or alkanolamine ion,

sulfonates of unsaturated fatty acids containing from about 8 to about 24 C atoms and from about 1 to about 6 double bonds,
esters of tartaric acid and citric acid with alcohols, the addition products of approximately 2 to about 15 molecules of ethylene oxide and/or propylene oxide onto fatty alcohols containing from about 8 to about 22 C atoms, and/or
alkyl- and/or alkenyl ether phosphates with formula

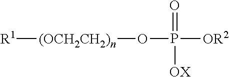

in which R¹ suitably represents an aliphatic hydrocarbon residue containing from about 8 to about 30 carbon atoms, R² represents hydrogen, a residue (CH₂CH₂O)ₙR¹ or X, n represents numbers from about 0 to about 10 and X represents hydrogen, an alkali metal or alkaline earth metal or the group —NR³R⁴R⁵R⁶, wherein R³ to R⁶, independently of each other, represent a C₁ to C₄ hydrocarbon residue.

Preferred anionic surfactants are alkyl sulfates and/or alkylether sulfate salts with formula R—(OCH₂—CH₂)ₙ—O—SO₃X, in which R preferably represents a straight-chained or branched, saturated or mono- or poly-unsaturated alkyl- or alkenyl residue containing from about 8 to about 24 carbon atoms, n represents 0 or from about 1 to about 12 and X represents an alkali, alkaline earth, ammonium or alkanolamine ion.

Suitable anionic surfactants e) are straight-chained or branched alkylether sulfate salts with the aforementioned formula, which contain an alkyl residue R containing from about 8 to about 18 and in particular from about 10 to about 16 C atoms as well as from about 1 to about 6 and for example from about 2 to about 4 ethylene oxide units.

The sodium, magnesium and/or triethanolamine salts of linear or branched lauryl, tridecyl and/or myristyl sulfates, which have a degree of ethoxylation of from about 2 to about 4, are suitable.

Surfactants known by their INCI name of sodium laureth sulfate are suitable.

The proportion by weight of the anionic surfactant e) with respect to the total weight of the hair treatment agent (shampoo) as contemplated herein is suitably from about 1.00 to about 15.00% by weight.

A proportion by weight of from about 2.00 to about 14.00% by weight, for example of from about 3.00 to about 13.00% by weight and in particular of from about 5.00 to about 12.50% by weight is used in an embodiment.

Examples of amphoteric and/or zwitterionic surfactants f) which may be used in the hair treatment agents (shampoos) as contemplated herein include one or more compounds with the following formulae (I) to (VII), in which the residue R respectively represents a straight-chained or branched, saturated or mono- or poly-unsaturated alkyl or alkenyl residue containing from about 7 to about 23 carbon atoms (formulae (I) and (II)) or represent a straight-chained or branched, saturated or mono- or poly-unsaturated alkyl or alkenyl residue containing from about 8 to about 24 carbon atoms (formulae (III) to (VII)):

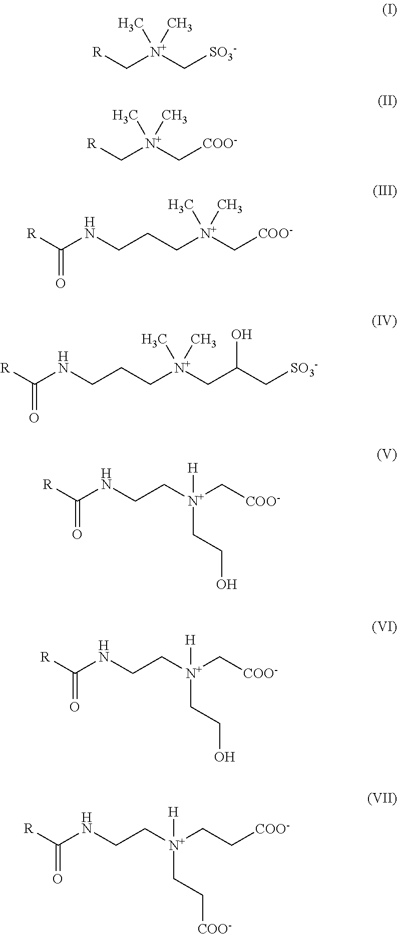

Amphoteric and/or zwitterionic surfactants with one of the aforementioned formulae (I) to (VII) primarily contain, as the residue R, a straight-chained or branched, saturated, mono- or poly-unsaturated alkyl residue containing from about 8 to about 20, for example from about 8 to about 18 and in particular containing from about 8 to about 16 C atoms.

Amphoteric and/or zwitterionic surfactants in which the residue R is derived from coconut oil are suitable.

Amphoteric/zwitterionic surfactants that are known by the INCI names sodium cocoamphoacetate, disodium cocoamphodiacetate, sodium lauroamphoacetate, sodium lauroamphodiacetate, sodium cocoamphopropionate, disodium cocoamphodipropionate, coco betaine, lauryl betaine, cocamidopropyl betaine and/or lauramidopropyl betaine and which are available from several suppliers are suitable.

Surfactants with the INCI names cocamidopropyl betaine, lauramidopropyl betaine, cocoampho(di)acetate and/or lauroapho(di)acetate are exemplary.

The proportion by weight of the at least one amphoteric and/or zwitterionic surfactant f) with respect to the total weight of the hair treatment agent (shampoo) as contemplated herein is preferably from about 1.00 to about 10.00% by weight. A proportion by weight of from about 1.25 to about 8.00% by weight, for example of from about 1.50 to about 7.50% by weight, for example from about 1.75 to about 6.00% by weight and in particular from about 2.00 to about 5.00% by weight is exemplary.

Hair treatment agents as contemplated herein that are formulated as anti-dandruff hair tonic have a pH in the range from about 4.0 to about 8.5, for example from about 5.0 to about 8.0, for example from about 6.0 to about 7.5 and in particular from about 6.5 to about 7.5.

In a further embodiment, for the purposes of stabilization and/or in order to obtain optimal foaming properties and mildness (in the event that the hair treatment agent as contemplated herein has been formulated as a hair shampoo), the hair treatment agents as contemplated herein may contain at least one non-ionic surfactant and/or non-ionic emulsifying agent.

The proportion by weight of the non-ionic surfactant and/or of the non-ionic emulsifying agent with respect to the total weight of the hair treatment agent as contemplated herein is suitably from about 0.01 to about 3.00% by weight, for example from about 0.02 to about 2.50% by weight, for example from about 0.03 to about 2.00% by weight and in an embodiment from about 0.05 to about 1.50% by weight.

Suitable non-ionic surfactants/emulsifying agents as contemplated herein include the following:
addition products of from about 4 to about 30 mol ethylene oxide and/or from about 0 to about 5 mol propylene oxide onto linear fatty alcohols containing from about 8 to about 22 C atoms, onto fatty acids containing from about 12 to about 22 C atoms and onto alkyl phenols containing from about 8 to about 15 C atoms in the alkyl group,
addition products of ethylene oxide and polyglycerine onto methylglucoside fatty acid esters, fatty acid alkanolamides and fatty acid glucamides,

C₈-C₃₀ fatty acid mono- and -diesters of addition products of from about 1 to about 30 mol ethylene oxide onto glycerine,

amine oxides,

sorbitan fatty acid esters and addition products of ethylene oxide onto sorbitan fatty acid esters such as polysorbates, for example,

fatty acid alkanolamides with the following general formula:

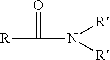

in which R suitably represents a linear or branched, saturated or unsaturated alkyl or alkenyl residue containing from about 8 to about 24 carbon atoms and the residues R′ represent hydrogen or the group —(CH₂)ₙOH, in which n represents the numbers from about 2 or about 3, with the proviso that at least one of the residues R′ represents the aforementioned residue —(CH₂)ₙOH,

fatty acid esters of sugars and addition products of ethylene oxide onto fatty acid esters of sugar,

addition products of ethylene oxide onto fatty acid alkanolamides and fatty amines,

alkyl(oligo)glucosides,

mixtures of alkyl(oligo)glucosides and fatty alcohols, for example the commercially available product Montanov®68,
addition products of from about 5 to about 60 mol ethylene oxide onto castor oil and hydrogenated castor oil,
partial esters of polyols containing from about 3 to about 6 carbon atoms with saturated fatty acids containing from about 8 to about 22 C atoms,
sterols. “Sterols” should be understood to mean a group of steroids which carry a hydroxy group on C3 of the steroid backbone and are isolated both from animal tissue (zoosterols) and also from vegetable fats (phytosterols). Examples of zoosterols are cholesterol and lanosterol. Examples of suitable phytosterols are ergosterol, stigmasterol and sitosterol. Sterols are also isolated from fungi and yeasts; they are known as mycosterols.
phospholipids. This primarily means glucose-phospolipids which are obtained, for example, as lecithins or phosphatidylcholines from egg yolk or from plant seeds (for example soyabeans).

Suitable alkyl(oligo)glycosides may be selected from compounds with general formula RO-[G]ₓ, in which [G] preferably derives from aldoses and/or ketoses containing from about 5 to about 6 carbon atoms, preferably from glucose.

The index x represents the degree of oligomerization (DO), i.e. the distribution of the mono- and oligo-glycosides. The index x preferably has a value in the range from about 1 to about 10, for example in the range from about 1 to about 3, wherein in this regard, it does not have to be a whole number but may be a fractional number which can be determined analytically.

Suitable alkyl(oligo)glycosides have a degree of oligomerization of between from about 1.2 and about 1.5.

The residue R suitably represents at least one alkyl- and/or alkenyl residue containing from about 4 to about 24 C atoms.

Suitable alkyl(oligo)glycosides are those compounds known by the INCI names caprylyl/capryl glucoside, decyl glucoside, lauryl glucoside and coco glucoside.

Suitable amine oxides may be selected from at least one compound with general formula (I) or (II):

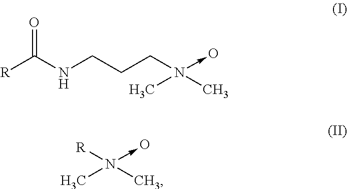

in which R respectively represents a linear or branched, saturated or mono- or poly-unsaturated alkyl or alkenyl residue containing from about 6 to about 24 carbon atoms, for example containing from about 8 to about 18 carbon atoms.

Suitable surfactants are those with the aforementioned formulae (I) or (II) with the INCI names cocamine oxide, lauramine oxide and/or cocamidopropylamine oxide, which are commercially available from a number of different suppliers.

The term “suitable C₈-C₃₀ fatty acid mono- and di-esters of addition products of from about 1 to about 30 mol ethylene oxide onto glycerine” should preferably be understood to mean those with the INCI names PEG(1-10) glyceryl cocoate, in particular PEG-7 glyceryl cocoate.

It may be of further advantage to combine the ethoxylated fatty acid esters with further ethoxylated fatty acid esters. Product mixtures of this type are commercially available—for example with the designation “Antil 200®” (INCI name: PEG-200 hydrogenated glyceryl palmate, PEG-7 glyceryl cocoate) from Evonik.

Suitable non-ionic surfactants which may be contained in the compositions as contemplated herein are fatty acid alkanolamides, in particular those compounds known by the INCI names cocamide MEA and/or cocamide MIPA; alkyl(oligo)glucosides, in particular those compounds known by the INCI names caprylyl/capryl glucoside, decyl glucoside, lauryl glucoside and/or coco glucoside; C₈-C₃₀ fatty acid mono- and di-esters of addition products of from about 1 to about 30 mol ethylene oxide onto glycerine, in particular the compound known by the INCI name PEG-7 glyceryl cocoate; and/or addition products of from about 4 to about 30 mol ethylene oxide and/or from about 0 to about 5 mol propylene oxide onto linear fatty alcohols containing from about 8 to about 22 C atoms.

Furthermore, for specific embodiments, it may be advantageous for the hair treatment agent as contemplated herein to contain hydrogenated castor oil. It has been discovered that a hydrogenated castor oil content can contribute to further stabilization of the anti-dandruff complex as contemplated herein.

In an embodiment, therefore, the hair treatment agents as contemplated herein preferably contain—with respect to their total weight—from about 0.01 to about 2.00% by weight, for example from about 0.02 to about 1.50% by weight, for example from about 0.03 to about 1.00% by weight and in an embodiment from about 0.05 to about 0.50% by weight of hydrogenated castor oil (INCI name: hydrogenated castor oil).

In addition to the aforementioned included and optional substances a) to g), advantageously, the agents as contemplated herein further contain the scalp soothing substances h) and/or the scalp nurturing substances i). In particular, the state of a scalp that is already flaky, irritated, sensitive and/or injured can be improved when at least one substance from groups h) and/or i) is added to the hair treatment agents as contemplated herein.

In a further embodiment, the hair treatment agents as contemplated herein therefore additionally contain—with respect to their weight—from about 0.01 to about 3.00% by weight, for example from about 0.02 to about 2.50% by weight, for example from about 0.03 to about 2.00% by weight and in particular from about 0.05 to about 1.50% by weight of at least one scalp soothing substance h) and/or at least one scalp nurturing substance i).

Suitable substances h) may therefore be used in the hair treatment agents as contemplated herein—with respect to their weight—in a quantity of from about 0.01 to about 2.00% by weight, for example of from about 0.02 to about 1.50% by weight, for example of from about 0.03 to about 1.00% by weight and in an embodiment of from about 0.05 to about 0.75% by weight, and are selected from allantoin, alpha-bisabolol, alpha-liponic acid and/or glycyrrethinic acid, for example from allantoin and/or alpha-bisabolol.

Suitable substances h) may be selected from

cationic conditioning polymers

protein hydrolysates,

vitamins,

fats, oils and/or waxes, and/or

glycerine.

The term “suitable cationic conditioning polymers” should, for example, be understood to include:

quaternized cellulose polymers, especially polyquaternium 10, commercially obtainable under the trade names Celquat® and Polymer JR®,

hydrophobically modified cellulose derivatives, for example the cationic polymers marketed under the SofiCat® trade names,

cationic alkylpolyglycosides,

cationic honey, for example the commercial product Honeyquat® 50,

cationic guar derivatives, in particular such as the products marketed under the trade names Cosmedia®Guar N-Hance® and Jaguar®,

polymeric dimethyldiallylammonium salts and their copolymers with esters and amides of acrylic acid and methacrylic acid, especially polyquaternium 6 and polyquaternium 7. The commercially available products known by the designations Merquat®100 (poly(dimethyldiallylammonium chloride)) and Merquat®550 (dimethyldiallylammonium chloride-acrylamide copolymer) are examples of such cationic polymers,

copolymers of vinylpyrrolidone with quaternized derivatives of dialkylaminoalkyl acrylate and methacrylate, such as, for example, vinylpyrrolidone dimethylaminoethyl methacrylate copolymers quaternized with diethylsulfate. Compounds of this type are commercially available under the designations Gafquat®734 and Gafquat®755,

vinylpyrrolidone-vinylimidazolium methochloride copolymers, obtainable under the designations Luviquat® FC 370, FC 550, FC 905 and HM 552,

quaternized polyvinylalcohol,

as well as the polymers known by the designations

polyquaternium 2, polyquaternium 17, polyquaternium 18, polyquaternium 24, polyquaternium 27, polyquaternium 32, polyquaternium 37, polyquaternium 74 and polyquaternium 89.

Suitable cationic polymers are quaternized cellulose polymers, hydrophobically modified quaternized cellulose polymers, cationic guar derivatives and/or cationic polymers based on acrylic acid (derivatives), which are for example selected from the polymers known by the INCI names guar hydroxypropyltrimonium chloride, polyquaternium 4, polyquaternium 6, polyquaternium 7, polyquaternium 10, polyquaternium 37, polyquaternium 67 and/or polyquaternium 72.

Cationic polysaccharide polymers are suitable; the aforementioned quaternized cellulose polymers are used in an embodiment.

The proportion by weight of the cationic conditioning polymer(s) with respect to the total weight of the hair treatment agent as contemplated herein is for example from about 0.01 to about 2% by weight, for example from about 0.05 to about 1.50% by weight and in one embodiment from about 0.10 to about 1.00% by weight.

The term “protein hydrolysates” should be understood to mean mixtures of products that may be obtained by acidic, basic or enzymatically catalyzed digestion of proteins. Protein hydrolysates of plant, animal and/or marine origin may be used.

Examples of protein hydrolysates are elastin, collagen, keratin, silk and milk protein hydrolysates, which may also be in the form of salts. Such products are, for example, marketed under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydrolysates of plant origin, for example soya, almond, rice, pea, potato and wheat protein hydrolysates, are preferred. Such products are, for example, obtainable under the trade names Gluadin® (Cognis), DiaMin® (Diamalt), Lexein® (Inolex) and Crotein® (Croda).

It is also possible to use cationic protein hydrolysates, wherein the basic protein hydrolysate may be derived from animals, for example from collagen, milk or keratin, from the plant, for example from wheat, maize, rice, potatoes, soya or almonds, from marine life forms, for example from fish collagen or algae, or from protein hydrolysates which are obtained biotechnologically. The protein hydrolysates forming the basis of the cationic derivatives may also be obtained from the corresponding proteins by employing a chemical process, in particular alkaline or acidic hydrolysis, by an enzymatic hydrolysis and/or a combination of both types of hydrolysis. The hydrolysis of proteins usually produces a protein hydrolysate with a molecular weight distribution of approximately 100 Dalton up to several thousand Dalton. Those cationic protein hydrolysates with a base protein fraction with a molecular weight of from about 100 up to about 25000 Dalton, preferably from about 250 to about 5000 Dalton, are preferred. Furthermore, the term “cationic protein hydrolysates” should also be understood to include quaternary amino acids and mixtures thereof. The quaternization of the protein hydrolysates or the amino acids is often carried out using quaternary ammonium salts such as, for example, N,N-dimethyl-N-(n-alkyl)-N-(2-hydroxy-3-chloro-n-propyl)-ammonium halides. Furthermore, the cationic protein hydrolysates may also be derivatized even further. Typical examples of cationic protein hydrolysates and derivatives and their INCI names and which are commercially available are as follows: cocodimonium hydroxypropyl hydrolyzed collagen, cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether HCl, hydroxypropyltrimonium gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratin, hydroxypropyltrimonium hydrolyzed rice bran protein, hydroxyproypltrimonium hydrolyzed silk, hydroxypropyltrimonium hydrolyzed soy protein, hydroxypropyl hydrolyzed vegetable protein, hydroxypropyltrimonium hydrolyzed wheat protein, hydroxypropyltrimonium hydrolyzed wheat protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, laurdimonium hydroxypropyl hydrolyzed casein, laurdimonium hydroxypropyl hydrolyzed collagen, laurdimonium hydroxypropyl hydrolyzed keratin, laurdimonium hydroxypropyl hydrolyzed silk, laurdimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratin, steardimonium hydroxypropyl hydrolyzed rice protein, steardimonium hydroxypropyl hydrolyzed silk, steardimonium hydroxypropyl hydrolyzed soy protein, steardimonium hydroxypropyl hydrolyzed vegetable protein, steardimonium hydroxypropyl hydrolyzed wheat protein, steartrimonium hydroxyethyl hydrolyzed collagen, quaternium-76 hydrolyzed collagen, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, quaternium-79 hydrolyzed wheat protein.

The proportion by weight of the protein hydrolysate(s) with respect to the total weight of the hair treatment agent is for example from about 0.01 to about 3% by weight, for example from about 0.025 to about 2% by weight and in an embodiment from about 0.05 to about 1% by weight.

The term “suitable vitamins” should be understood to mean the following vitamins, provitamins and vitamin precursors:

Vitamin A: substances described as belonging to the vitamin A group are retinol (vitamin A₁) as well as 3,4-didehydroretinol (vitamin A₂). β-carotene is the provitamin for retinols. Examples of vitamin A components are vitamin A acids and their esters, vitamin A aldehyde and vitamin A alcohol, as well as their esters such as the palmitate and the acetate.
Vitamin B: the vitamin B group or the vitamin B complex includes, inter alia:

vitamin B₁ (thiamine)

vitamin B₂ (riboflavin)

vitamin B₃. This description usually includes the compounds nicotinic acid and nicotinic acid amide (niacinamide)

vitamin B₅ (pantothenic acid and panthenol). In the context of this group, panthenol is preferably used. Particular derivatives of panthenol are the esters and ethers of panthenol, pantolactone as well as cationically derivatized panthenols. Individual examples are panthenol triacetate, panthenol monoethylether and its monoacetate, as well as cationic panthenol derivatives.

vitamin B₆ (pyridoxine as well as pyridoxamine and pyridoxal).

Vitamin C (ascorbic acid): use in the form of palmitic acid esters, glucosides or phosphates may be preferable. Use in combination with tocopherols may also be preferred.
Vitamin E (tocopherols, in particular α-tocopherol).
Vitamin F: the term “vitamin F” should usually be understood to mean fatty acids, in particular linoleic acid, linolenic acid and arachidonic acid.
Vitamin H: the compound (3aS.4S, 6aR)-2-oxohexahydrothienol[3,4-d]-imidazol-4-valerian acid is described as vitamin H; it is now known by the trivial name of biotin.

Vitamins, provitamins and vitamin precursors from groups A, B, E and H are suitable. Nicotinic acid amide, biotin, pantolactone and/or panthenol are suitable.

The proportion by weight of the vitamin(s), vitamin derivative(s), provitamin(s) and/or vitamin precursor(s) with respect to the total weight of the hair treatment agents is for example from about 0.001% to about 2% by weight, for example from about 0.005% to about 1% by weight and in an embodiment from about 0.01% to about 0.5% by weight.

Suitable fats, oils and/or waxes that may be used in the hair treatment agents as contemplated herein are preferably of mineral, natural and synthetic origin.

Triglycerides and mixtures of triglycerides are usually employed as the natural (plant) oils. Preferred natural oils are coconut oil, (sweet) almond oil, walnut oil, peach kernel oil, apricot kernel oil, avocado oil, tea tree oil soya oil, sesame oil, sunflower oil, tsubaki oil, evening primrose oil, rice bran oil, palm kernel oil, mango kernel oil, meadowfoam seed oil, thistle oil, macadamia nut oil, grapeseed oil, amaranthus seed oil, argan oil, bamboo oil, olive oil, wheatgerm oil, pumpkin seed oil, mallow oil, hazelnut oil, safflower oil, canola oil, sasanqua oil, jojoba oil, rambutan seed oil, cocoa butter and shea butter.

Mineral oils, paraffin and isoparaffin oils as well as synthetic hydrocarbons are used as the mineral oils. An example of a hydrocarbon that may be used is the commercially available product 1,3-di-(2-ethylhexyl)-cyclohexane (Cetiol® S).

Furthermore, a dialkylether may act as the oil component.

In an embodiment, dialkylethers that may be used are di-n-alkylethers containing a total of from about 12 to about 36 C atoms, in particular from about 12 to about 24 C atoms such as, for example, di-n-octylether, di-n-decylether, di-n-nonylether, di-n-undecylether, di-n-dodecylether, n-hexyl-n-octylether, n-octyl-n-decylether, n-decyl-n-undecylether, n-undecyl-n-dodecylether and n-hexyl-n-undecylether as well as di-tert-butylether, di-iso-pentylether, di-3-ethyldecylether, tert-butyl-n-octylether, iso-pentyl-n-octylether and 2-methylpentyl-n-octylether.

Di-n-octylether is suitable; it is commercially available under the trade name Cetiol® OE.

Silicone compounds are the exemplary synthetic oils.

Silicones have excellent conditioning properties on hair. In particular, in many cases they have a positive effect on the feel and softness of the hair.

It is therefore desirable to use silicones in cosmetic hair treatment agents. Suitable silicones may be selected from among:

a) polyalkylsiloxanes, polyarylsiloxanes, polyalkylarylsiloxanes, which are volatile or non-volatile, straight-chained, branched or cyclic, cross-linked or not cross-linked;
b) polysiloxanes, which contain one or more organofunctional groups in their general structure, which are selected from:
(i) substituted or unsubstituted amino groups;
(ii) (per)fluorinated groups;
(iii) thiol groups;
(iv) carboxylate groups;
(v) hydroxy groups;
(vi) alkoxyl groups;
(vii) acyloxyalkyl groups;
(viii) amphoteric groups;
(ix) bisulfite groups;
(x) hydroxyacylamino groups;
(xi) carboxy groups;
(xii) sulfonic acid groups; and
(xiii) sulfate or thiosulfate groups;
c) linear polysiloxane(A)-polyoxyalkylene(B)-block copolymers of type (A-B)n with n>3;
d) grafted silicone polymers with non-silicone-containing, organic backbones, which consist of an organic main chain formed from organic monomers which do not contain any silicone onto which at least one polysiloxane macromer is grafted within the chain as well as, if appropriate, onto at least one chain end;
e) grafted silicone polymers with polysiloxane backbones onto which non-silicone-containing organic monomers have been grafted, which have a polysiloxane main chain onto which at least one organic macromer which does not contain any silicone has been grafted within the chain as well as, if appropriate, onto at least one end thereof,
f) or mixtures thereof.

The term “fats” should be understood to mean fatty alcohols as well as natural and synthetic waxes which may be present both in the solid form and also in the liquid form in aqueous dispersion.

The fatty alcohols that may be used are saturated, mono- or poly-unsaturated branched or unbranched fatty alcohols containing C₆-C₃₀, preferably C₁₀-C₂₂ and for example C₁₂-C₂₂ carbon atoms. Examples that may be used are decanol, octanol, octenol, dodecenol, decenol, octadienol, dodecadienol, decadienol, oleyl alcohol, erucic alcohol, ricinoleic alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as their Guerbet alcohols; this list is by way of non-limiting example. However, the fatty alcohols preferably derive from natural fatty acids, usually obtained by starting from the esters of the fatty acids by reduction. Those fatty alcohol cuts that are obtained by reduction of naturally occurring triglycerides such as beef suet, palm oil, peanut oil, beet oil, cotton seed oil, soya oil, sunflower seed oil and linseed oil or fatty acid esters produced from their transesterification products with the corresponding alcohols, and thus constitute a mixture of different fatty alcohols, may also be used in the context of the present disclosure. Substances of this type are commercially available, for example, under the trade names Stenol®, for example Stenol® 1618 or Lanette®, for example Lanette® O or Lorol®, for example Loror C8, Lorol® C14, Lora′ C18, Lorol® C8-18, HD-Ocenol®, Crodacol®, for example Crodacol® CS, Novol®, Eutanol® G, Guerbitor® 16, Guerbitol® 18, Guerbitol® 20, Isofol® 12, Isofol® 16, Isofol® 24, Isofol® 36, Isocarb® 12, Isocarb® 16 or Isocarb® 24. Clearly, it is also possible to use lanolin alcohols such as those which are commercially available under the designations Corona®, White Swan®, Coronet® or Fluilan®.

Solid paraffins or isoparaffins, carnauba waxes, beeswaxes, candelilla waxes, ozokerite, ceresin, spermaceti, sunflower wax, fruit waxes such as apple wax or citrus wax, for example, or microwaxes formed from PE or PP, may be used as natural or synthetic waxes. Waxes of this type are available, for example, from Kahl & Co., Trittau.

Further examples of fats are:

ester oils. The term “ester oils” should be understood to mean esters of C₆-C₃₀ fatty acids containing C₂-C₃₀ fatty alcohols. Monoesters of fatty acids with alcohols containing from about 2 to about 24 C atoms are preferred. Examples of fatty acid fractions in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselenic acid, linoleic acid, linolenic acid, eleostearic acid, arachidic acid, gadoleic acid, behenic acid and erucic acid, as well as technical mixtures thereof.

Examples of the fatty alcohol fractions in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselenyl alcohol, linolyl alcohol, linolenyl alcohol, eleostearyl alcohol, arachidyl alcohol, gadoleyl alcohol, behenyl alcohol, erucyl alcohol and brassidyl alcohol, as well as technical mixtures thereof. Isopropylmyristate (Rilanit® IPM), isononanoic acid-C16-18-alkylester (Cetiol® SN), 2-ethylhexylpalmitate (Cegesoft® 24), stearic acid-2-ethylhexylester (Cetiol® 868), cetyloleate, glycerine tricaprylate, coconut oil alcohol caprinate/-caprylate (Cetiol® LC), n-butyl stearate, oleyl erucate (Cetiol® J 600), isopropyl palmitate (Rilanit® IPP), oleyl oleate (Cetiol®), lauric acid hexylester (Cetiol® A), di-n-butyladipate (Cetiol® B), myristyl myristate (Cetiol® MM), cetearyl isononanoate (Cetiol® SN), oleic acid decylester (Cetiol® V) are suitable.

dicarboxylic acid esters such as di-n-butyladipate, di-(2-ethylhexyl)-adipate, di-(2-ethylhexyl)-succinate and di-isotridecyl acetate as well as diol esters such as ethylene glycol dioleate, ethylene glycol diisotridecanoate, propyleneglycol-di(2-ethylhexanoate), propylene glycol diisostearate, propylene glycol dipelargonate, butanediol diisostearate, neopentyl glycol dicaprylate,

symmetrical, unsymmetrical or cyclic esters of carbonic acid with fatty alcohols,

glycerine carbonate or dicaprylyl carbonate (Cetiol® CC),

ethoxylated or non-ethoxylated mono-, di- and tri-fatty acid esters of saturated and/or unsaturated linear and/or branched fatty acids with glycerin, such as, for example, Monomuls® 90-O18, Monomuls® 90-L12, Cetiol® HE or Cutina® MD.

The proportion by weight of the oil, wax and/or fat components with respect to the total weight of the hair treatment agent as contemplated herein is for example from about 0.01 to about 3.00% by weight, for example from about 0.025 to about 2.50% by weight and in an embodiment from about 0.05 to about 2.00% by weight.

Glycerine may be added separately to the hair treatment agents as contemplated herein in a quantity of up to about 10% by weight (with respect to the total weight of the agent). However, it may also be a component of the aforementioned aqueous alcoholic support.

In a further embodiment, the hair treatment agents as contemplated herein contain, as the at least one scalp soothing substance h) and/or as the at least one nurturing substance i), panthenol, allantoin, alpha-bisabolol, a vegetable oil and/or wax, a cationic polymer and/or glycerine.

In this embodiment, for example, the hair treatment agent as contemplated herein contains at least one substance h) or i) selected from the group which is formed by panthenol, allantoin, alpha-bisabolol, polyquaternium 10, apricot kernel oil and/or jojoba oil.

Examples of other substances, auxiliary substances and additional substances which may preferably be contained in the hair treatment agents as contemplated herein are as follows:

plant extracts,

humectants,

fragrances,

UV filters,

thickeners such as gelatins or vegetable gums, for example agar-agar, guar gum, alginates, xanthan gum, gum arabicum, karaya gum, carob bean gum, linseed gums, dextrans, cellulose derivatives, for example methyl cellulose, hydroxyalkylcellulose and carboxymethyl cellulose, starch fractions and derivatives such as amylose, amylopectin and dextrins, clays and phyllosilicates such as bentonite, for example, or completely synthetic hydrocolloids such as polyvinyl alcohol, for example,

thickeners such as acrylic and methacrylic (co)polymers, for example the crosslinked homopolymers of acrylic acid (INCI name: carbomer), which are also known as carboxyvinylpolymers. Polyacrylic acids of this type are available, inter alia, from 3V Sigma under the trade names Polygel®, for example Polygel DA, and from B. F. Goodrich under the Carbopol® trade names, for example Carbopol 940 (molecular weight approximately 4000000), Carbopol 941 (molecular weight approximately 1250000) or Carbopol 934 (molecular weight approximately 3000000). Further suitable examples of acrylic acid copolymers are as follows:

copolymers of two or more monomers from the group formed by acrylic acid, methacrylic acid and their monoesters, preferably formed with C1-C4 alkanols (INCI name: acrylates copolymer), examples of which are copolymers of methacrylic acid, butyl acrylate and methyl methacrylate or butyl acrylate and methyl methacrylate and which are available, for example, from Rohm & Haas under the trade names Aculyn® and Acusol® as well as from Degussa (Goldschmidt) under the trade names Tego® Polymer, for example Aculyn 22, Aculyn 28, Aculyn 33 (crosslinked), Acusol 810, Acusol 820, Acusol 823 and Acusol 830;

crosslinked high molecular weight acrylic acid copolymers, examples of which are copolymers of C10-C30 alkyl acrylates crosslinked with an allyl ether of saccharose or of pentaerythritol with one or more monomers from the group formed by acrylic acid, methacrylic acid and their monoesters, preferably formed with C1-C4-alkanols (INCI name: acrylates-(C10-C30) alkyl acrylate crosspolymer) and which are available, for example, from B. F. Goodrich under the trade names Carbopol®, for example Carbopol ETD 2020 and Carbopol 1382 (INCI: acrylates-(C10-C30)-alkyl acrylate crosspolymer) as well as Carbopol Aqua 30,

structuring agents such as maleic acid and lactic acid,

colorants in order to color the agent,

pH-adjusting substances, for example α- and β-hydroxycarboxylic acids such as citric acid, lactic acid, malic acid, glycolic acid and/or bases such as alkanolamine and/or sodium hydroxide,

chelating agents such as EDTA, NTA, β-alanine diacetic acid and phosphonic acids,

ceramides. The term “ceramides” should be understood to mean N-acylsphingosine (fatty acid amide of sphingosine) or synthetic analogues of such lipids (what are known as pseudo-ceramides),

propellants such as propane-butane mixtures, N₂O, dimethylether, CO₂ and air,

antioxidants,

preservatives such as sodium benzoate or salicylic acid, for example,

additional viscosity regulators such as salts (NaCl).

In a second aspect, the present disclosure concerns the cosmetic use of a combination of substances containing:

a) from about 0.05 to about 5.0% by weight of zinc oxide,
b) from about 0.05 to about 3.00% by weight of at least one anti-dandruff substance that is different from a),
c) from about 0.10 to about 10.00% by weight of at least one saturated or unsaturated, straight-chained or branched C₁₀-C₂₄ carboxylic acid, and
d) from about 0.01 to about 5.00% by weight of at least one amino acid, in order to improve the anti-dandruff effectiveness of hair treatment agents, wherein the quantities given are with respect to the total weight of the hair treatment agent.

In a third aspect, the present disclosure provides a cosmetic method for removing, reducing, alleviating and/or preventing dandruff, in which a hair treatment agent as contemplated herein is applied to hair that is preferably wet, massaged in and then rinsed out with water after a treatment time of from about 5 seconds to about 5 minutes.

The statements made with regard to the hair treatment agents as contemplated herein apply correspondingly to the embodiments of the use as contemplated herein and the method as contemplated herein.

### Examples

The following hair treatment agents as contemplated herein were produced. The quantities in the table are given as a % by weight:

| | 1 | 2 | 3 | 4 |
|---|---|---|---|---|
| Zinc oxide | 0.05-5.00 | 0.10-4.00 | 0.25-3.00 | 0.50-2.00 |
| Anti-dandruff substance ≠ ZnO | 0.05-3.00 | 0.10-2.50 | 0.15-2.00 | 0.20-1.50 |
| C₁₀-C₂₄ carboxylic acid* | 0.10-10.00 | 0.50-8.00 | 1.00-6.00 | 1.50-5.00 |
| Amino acid | 0.01-5.00 | 0.02-4.00 | 0.05-3.00 | 0.10-2.00 |
| Aqueous or aqueous alcoholic | ad 100 | ad 100 | ad 100 | ad 100 |
| support and optional further | | | | |
| auxiliary and additional | | | | |
| substances | | | | |
| | 5 | 6 | 7 | 8 |
| Zinc oxide | 0.05-5.00 | 0.10-4.00 | 0.25-3.00 | 0.50-2.00 |
| Piroctone olamine and/or | 0.05-3.00 | 0.10-2.50 | 0.15-2.00 | 0.20-1.50 |
| climbazole | | | | |
| C₁₀-C₂₄ carboxylic acid* | 0.10-10.00 | 0.50-8.00 | 1.00-6.00 | 1.50-5.00 |
| Amino acid | 0.01-5.00 | 0.02-4.00 | 0.05-3.00 | 0.10-2.00 |
| Aqueous or aqueous alcoholic | ad 100 | ad 100 | ad 100 | ad 100 |
| support and optional further | | | | |
| auxiliary and additional | | | | |
| substances | | | | |
| | 9 | 10 | 11 | 12 |
| Zinc oxide | 0.05-5.00 | 0.10-4.00 | 0.25-3.00 | 0.50-2.00 |
| Anti-dandruff substance ≠ ZnO | 0.05-3.00 | 0.10-2.50 | 0.15-2.00 | 0.20-1.50 |
| Palmitic acid and/or stearic | 0.10-10.00 | 0.50-8.00 | 1.00-6.00 | 1.50-5.00 |
| acid | | | | |
| Amino acid | 0.01-5.00 | 0.02-4.00 | 0.05-3.00 | 0.10-2.00 |
| Aqueous or aqueous alcoholic | ad 100 | ad 100 | ad 100 | ad 100 |
| support and optional further | | | | |
| auxiliary and additional | | | | |
| substances | | | | |
| | 13 | 14 | 15 | 16 |
| Zinc oxide | 0.05-5.00 | 0.10-4.00 | 0.25-3.00 | 0.50-2.00 |
| Anti-dandruff substance ≠ ZnO | 0.05-3.00 | 0.10-2.50 | 0.15-2.00 | 0.20-1.50 |
| C₁₀-C₂₄ carboxylic acid* | 0.10-10.00 | 0.50-8.00 | 1.00-6.00 | 1.50-5.00 |
| Alanine, aspartic acid, | 0.01-5.00 | 0.02-4.00 | 0.05-3.00 | 0.10-2.00 |
| cysteine, cystine, glutamic | | | | |
| acid, glycine, proline, serine | | | | |
| and/or tyrosine; in particular | | | | |
| glycine | | | | |
| Aqueous or aqueous alcoholic | ad 100 | ad 100 | ad 100 | ad 100 |
| support and optional further | | | | |
| auxiliary and additional | | | | |
| substances | | | | |
| | 17 | 18 | 19 | 20 |
| Zinc oxide | 0.05-5.00 | 0.10-4.00 | 0.25-3.00 | 0.50-2.00 |
| Piroctone olamine and/or | 0.05-3.00 | 0.10-2.50 | 0.15-2.00 | 0.20-1.50 |
| climbazole | | | | |
| Palmitic acid and/or stearic | 0.10-10.00 | 0.50-8.00 | 1.00-6.00 | 1.50-5.00 |
| acid | | | | |
| Alanine, aspartic acid, | 0.01-5.00 | 0.02-4.00 | 0.05-3.00 | 0.10-2.00 |
| cysteine, cystine, glutamic | | | | |
| acid, glycine, proline, serine | | | | |
| and/or tyrosine | | | | |
| Aqueous or aqueous alcoholic | ad 100 | ad 100 | ad 100 | ad 100 |
| support and optional further | | | | |
| auxiliary and additional | | | | |
| substances | | | | |
| | 21 | 22 | 23 | 24 |
| Zinc oxide | 0.05-5.00 | 0.10-4.00 | 0.25-3.00 | 0.50-2.00 |
| Anti-dandruff substance ≠ ZnO | 0.05-3.00 | 0.10-2.50 | 0.15-2.00 | 0.20-1.50 |
| C₁₀-C₂₄ carboxylic acid* | 0.10-10.00 | 0.50-8.00 | 1.00-6.00 | 1.50-5.00 |
| Amino acid | 0.01-5.00 | 0.02-4.00 | 0.05-3.00 | 0.10-2.00 |
| Surfactant | max. 20.00 | max. 18.00 | max. 16.00 | max. 15.00 |
| Aqueous or aqueous alcoholic | ad 100 | ad 100 | ad 100 | ad 100 |
| support and optional further | | | | |
| auxiliary and additional | | | | |
| substances | | | | |
| | 25 | 26 | 27 | 28 |
| Zinc oxide | 0.05-5.00 | 0.10-4.00 | 0.25-3.00 | 0.50-2.00 |
| Anti-dandruff substance ≠ ZnO | 0.05-3.00 | 0.10-2.50 | 0.15-2.00 | 0.20-1.50 |
| C₁₀-C₂₄ carboxylic acid* | 0.10-10.00 | 0.50-8.00 | 1.00-6.00 | 1.50-5.00 |
| Amino acid | 0.01-5.00 | 0.02-4.00 | 0.05-3.00 | 0.10-2.00 |
| Anionic surfactant | 1.00-15.00 | 2.00-14.00 | 3.00-13.00 | 5.00-15.00 |
| Amphoteric surfactant | 1.00-10.00 | 1.25-8.00 | 1.50-7.50 | 1.75-6.00 |
| Aqueous or aqueous alcoholic | ad 100 | ad 100 | ad 100 | ad 100 |
| support and optional further | | | | |
| auxiliary and additional | | | | |
| substances | | | | |
| | 29 | 30 | 31 | 32 |
| Zinc oxide | 0.05-5.00 | 0.10-4.00 | 0.25-3.00 | 0.50-2.00 |
| Anti-dandruff substance ≠ ZnO | 0.05-3.00 | 0.10-2.50 | 0.15-2.00 | 0.20-1.50 |
| C₁₀-C₂₄ carboxylic acid* | 0.10-10.00 | 0.50-8.00 | 1.00-6.00 | 1.50-5.00 |
| Amino acid | 0.01-5.00 | 0.02-4.00 | 0.05-3.00 | 0.10-2.00 |
| Anionic surfactant | 1.00-15.00 | 2.00-14.00 | 3.00-13.00 | 5.00-15.00 |
| Amphoteric surfactant | 1.00-10.00 | 1.25-8.00 | 1.50-7.50 | 1.75-6.00 |
| Hydrogenated castor oil | 0.01-2.00 | 0.02-1.50 | 0.03-1.00 | 0.05-0.50 |
| Aqueous or aqueous alcoholic | ad 100 | ad 100 | ad 100 | ad 100 |
| support and optional further | | | | |
| auxiliary and additional | | | | |
| substances | | | | |
| | 33 | 34 | 35 | 36 |
| Zinc oxide | 0.05-5.00 | 0.10-4.00 | 0.25-3.00 | 0.50-2.00 |
| Anti-dandruff substance ≠ ZnO | 0.05-3.00 | 0.10-2.50 | 0.15-2.00 | 0.20-1.50 |
| C₁₀-C₂₄ carboxylic acid* | 0.10-10.00 | 0.50-8.00 | 1.00-6.00 | 1.50-5.00 |
| Amino acid | 0.01-5.00 | 0.02-4.00 | 0.05-3.00 | 0.10-2.00 |
| Anionic surfactant | 1.00-15.00 | 2.00-14.00 | 3.00-13.00 | 5.00-15.00 |
| Amphoteric surfactant | 1.00-10.00 | 1.25-8.00 | 1.50-7.50 | 1.75-6.00 |
| Hydrogenated castor oil | 0.01-2.00 | 0.02-1.50 | 0.03-1.00 | 0.05-0.50 |
| Cationic polymer | 0.01-2.00 | 0.05-1.50 | 0.10-1.00 | 0.15-0.75 |
| Aqueous or aqueous alcoholic | ad 100 | ad 100 | ad 100 | ad 100 |
| support and optional further | | | | |
| auxiliary and additional | | | | |
| substances | | | | |

| | 37 | 38 | 39 | 40 |
|---|---|---|---|---|
| Zinc oxide | 1.10 | 1.10 | 1.10 | 1.10 |
| Piroctone olamine | 0.50 | 0.50 | 0.30 | |
| Climbazole | | | | 0.50 |
| Palmitic acid | 3.50 | 1.80 | 1.80 | |
| Stearic acid | | 1.80 | 1.80 | 3.50 |
| Glycine | 0.10 | 0.10 | | |
| Alanine | | | 0.10 | 0.10 |
| Sodium laureth sulfate | 10 | 10 | 10 | 10 |
| Cocamidopropyl betaine | 1.60 | 1.60 | 1.60 | |
| Disodium lauroamphodiacetate | | 1.20 | 1.20 | 2.40 |
| PEG-7 glyceryl cocoate | 0.70 | 0.70 | 0.70 | 0.70 |
| Hydrogenated castor oil | 0.20 | 0.20 | 0.20 | 0.20 |
| Cocamide MEA | 0.70 | 0.70 | | |
| Polyquaternium-10 | 0.20 | 0.20 | 0.20 | 0.20 |
| Sodium benzoate | 0.45 | 0.45 | 0.45 | 0.45 |
| Citric acid | | | | |
| Fragrance | 0.45 | 0.45 | 0.45 | 0.45 |
| Water | ad 100 | ad 100 | ad 100 | ad 100 |
| pH | 4.5-5.5 | 4.5-5.5 | 4.5-5.5 | 4.5-5.5 |

*saturated or unsaturated, straight-chained or branched

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair treatment agent comprising, in a cosmetic support:
a) from about 0.05 to about 5.0% by weight of zinc oxide,
b) from about 0.05 to about 3.00% by weight of at least one anti-dandruff substance that is different from a),
c) from about −0.10 to about 10.00% by weight of at least one saturated or unsaturated, straight-chained or branched C₁₀-C₂₄ carboxylic acid, and
d) from about 0.01 to about 5.00% by weight of at least one amino acid,
wherein the quantities given are with respect to the total weight of the hair treatment agent.

2. The hair treatment agent of claim 1, comprising—with respect to the total weight of the hair treatment agent—from about 0.10 to about 4.00% by weight of the zinc oxide.

3. The hair treatment agent of claim 1, wherein the at least one anti-dandruff substance b) is soluble in a surfactant solution and/or soluble in water, and is chosen from one or more of: piroctone olamine, climbazole, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts and/or arnica extracts.

4. The hair treatment agent of claim 3, wherein the at least one anti-dandruff substance b) is chosen from: piroctone olamine and/or climbazole.

5. The hair treatment agent of claim 1, comprising—with respect to the total weight of the hair treatment agent—from about 0.10 to about 2.50% by weight, of the at least one anti-dandruff substance b).

6. The hair treatment agent of claim 1, comprising as the component c) at least one saturated or unsaturated, straight-chained or branched carboxylic acid having a carbon chain comprising from about 12 to about 22.

7. The hair treatment agent of claim 1, comprising—with respect to the total weight of the hair treatment agent—from about 0.50 to about 8.00% by weight of the at least one saturated or unsaturated, straight-chained or branched C₁₀-C₂₄ carboxylic acid.

8. The hair treatment agent of claim 1, comprising as the component d) at least one non-essential amino acid chosen from: alanine, aspartic acid, cysteine, cystine, glutamic acid, glycine, proline, serine and/or tyrosine.

9. The hair treatment agent of claim 1, comprising—with respect to the total weight of the hair treatment agent—from about 0.02 to about 4.00% by weight of the at least one amino acid.

10. The hair treatment agent of claim 1, additionally comprising at least one anionic and at least one amphoteric and/or zwitterionic surfactant.

11. The hair treatment agent of claim 1, additionally comprising hydrogenated castor oil in an amount by weight of from about 0.01 to about 2.00% by weight with respect to the total weight of the hair treatment agent.

12. The hair treatment agent of claim 1, additionally comprising at least one cationic polymer.

13. The hair treatment agent of claim 1, wherein the hair treatment agent is in the form of a hair shampoo and has a pH in the range from about 4.0 to about 6.0.

14. (canceled)

15. (canceled)

16. The hair treatment agent of claim 1, wherein the hair treatment agent is in the form of a hair shampoo and has a pH in the range from about 4.2 to about 5.8.

17. The hair treatment agent of claim 1, wherein the hair treatment agent is in the form of a hair shampoo and has a pH in the range from about 4.3 to about 5.6.

18. The hair treatment agent of claim 1, wherein the hair treatment agent is in the form of a hair shampoo and has a pH in the range from about 4.5 to about 5.5.

19. The hair treatment agent of claim 1, comprising as the component d) at least one non-essential amino acid chosen from: glycine, proline and/or serine.

20. The hair treatment agent of claim 1, comprising as the component d) non-essential amino acid glycine.

21. A cosmetic method of using a hair treatment agent in order to provide anti-dandruff effectiveness, the hair treatment agent comprising:
a) from about 0.05 to about 5.0% by weight of zinc oxide,
b) from about 0.05 to about 3.00% by weight of at least one anti-dandruff substance that is different from a),
c) from about 0.10 to about 10.00% by weight of at least one saturated or unsaturated, straight-chained or branched C₁₀-C₂₄ carboxylic acid, and
d) from about 0.01 to about 5.00% by weight of at least one amino acid,
wherein the quantities are given with respect to the total weight of the hair treatment agent,
wherein the method comprises the step of applying the hair treatment agent to hair.

22. The cosmetic method of claim 21, wherein the step of applying comprises applying the hair treatment agent to hair that is wet, wherein the method further comprises the steps of massaging-in and then rinsing-out the hair treatment agent with water after a treatment time of from about 5 seconds to about 5 minutes
